(19) United States
(12) Patent Application Publication
(10) Pub. No.: US 2025/0034598 A1
(43) Pub. Date: Jan. 30, 2025

(54) SYSTEM AND METHOD FOR PRODUCING PRODUCT GAS COMPRISING METHANE

(71) Applicant: Nature Energy Biogas A/S, Odense SØ (DK)

(72) Inventors: Martin Dan JEPPESEN, Odense V (DK); Brian Dahl JØNSON, Odense SØ (DK); Lina Johanna Nåbo ANDERSEN, Morud (DK)

(73) Assignee: Nature Energy Biogas A/S, Odense SØ (DK)

(21) Appl. No.: 18/713,583

(22) PCT Filed: Nov. 29, 2022
(86) PCT No.: PCT/DK2022/050252

§ 371 Date: May 24, 2024

(30) Foreign Application Priority Data
Nov. 29, 2021 (WO) PCT/DK2021/050349

(51) Int. Cl. C12P 5/02; C12M 1/00; C12M 1/107; C12M 1/34; C12P 3/00

(52) U.S. Cl. CPC C12P 5/023; C12M 21/04; C12M 23/36; C12M 23/58; C12M 41/12; C12M 47/00; C12P 3/00

(57) ABSTRACT

A system for producing product gas comprising methane is disclosed, the system comprising a first inlet for receiving a first biomass, the first biomass having a dry matter content of at least 15% by weight of the first biomass, a chemical oxygen demand (COD) of at least 100,000 mg O2/L, and has a content of nitrogen of at least 4 gram per kg, a content of phosphorous of at least 5 gram per kg, and a content of potassium of at least 5 gram per kg, a second inlet for receiving a second biomass, a microbial electrolysis cell reactor for processing the first biomass to produce methane and/or hydrogen gas and a first biomass digestate, an anaerobic digestion reactor for processing the second biomass to produce methane and a second biomass digestate, a first output for discharging the first biomass digestate, a second output for discharging the second biomass digestate. Also, a method for producing product gas comprising methane is disclosed.

### FIELD OF INVENTION

The invention relates to a system and a method for producing product gas comprising methane, optionally also comprising hydrogen gas. Particularly, the invention relates to a system and a method according to the claims.

### BACKGROUND

Production of biogas from various biomass has become increasingly popular over the years. Biogas represents both an extra fuel gas and also a green alternative to fossil gas and other fossil fuels.

Therefore, a higher production of biogas is desirable, and much effort has been put into increasing the yield of biogas. For example, two step anaerobic digestion has shown to provide favorable biogas yield, as shown in WO 2020/099651 A1.

Also, there has been focus on increasing the possible usable biomass fractions, e.g. by increasing cultivation of suitable energy crops and/or by treating previously unusable fractions to become processable for biogas production.

Nevertheless, a need for increasing the biogas output still exists.

Also, obtaining valuable biproducts from the biogas production has gained increasing focus.

At the same time, other gasses e.g. for use as fuels are also becoming increasingly high in demand, e.g. hydrogen gas.

It is an object of the invention to solve the above problems and challenges.

### SUMMARY

The invention relates to a system for producing product gas comprising methane, the system comprising
a first inlet for receiving a first biomass, the first biomass having a dry matter content of at least 15% by weight of the first biomass and a chemical oxygen demand (COD) of at least 100,000 mg O2/L, the first biomass having at least one of
   a content of nitrogen of at least 4 gram per kg,
   a content of phosphorous of at least 5 gram per kg, and
   a content of potassium of at least 5 gram per kg,
a second inlet for receiving a second biomass,
a microbial electrolysis cell reactor for processing the first biomass to produce methane and/or hydrogen gas and a first biomass digestate,
an anaerobic digestion reactor for processing the second biomass to produce methane and a second biomass digestate,
a first output for discharging the first biomass digestate,
a second output for discharging the second biomass digestate.

Thus, according to the invention, the first biomass is processed separate from the second biomass.

An advantage of the present invention may be that by means of separate processing of the first biomass being a high energy fraction, as evident from the high COD, and also having high nutrient content, the second biomass may be processed in a more effective and/or cost-effective way due to the energy and nutrient content being within acceptable limits, such that e.g. costly dilution may be avoided, if the high energy first biomass is mixed with the second biomass and the high energy and/or nutrient content is to be avoided. Particularly, the anaerobic digestions reactor may typically include certain desirable microbial cultures, particularly methanogenic microbes, which however, may be undesirably influenced when the energy and/or and nutrient content is too high. Also, from an operational point of view, avoiding the inclusion of the first biomass into the anaerobic digestion of the second biomass may be advantageous due avoiding fluctuations in energy and nutrient content. Since the supply of the second biomass, which typically comprises industrial side streams such as dairy waste and molasses, typically may vary substantially both on amount and composition, mixing these into the second biomass may increase undesirable energy and nutrient content fluctuations.

Another advantage of the invention may be that separate digestate fractions are obtained, where the first biomass digestate has a higher P to N ratio than the second biomass digestate. Thereby, the system is not only advantageously designed for production of biogas and possibly hydrogen, but also for providing distinct digestate fractions, which can be mixed in a predetermined ratio to obtain fertilizer fractions of customized nutrient profiles, particularly N to P ratio.

A further advantage of the invention may be that by using a microbial electrolysis cell reactor for processing the first biomass, an effective processing of the first biomass may be obtained, since microbial electrolysis cell processing is relatively effective for processing biomass with high COD content, in contrast with anaerobic digestion, which may be less effective for high COD biomasses.

A further advantage of the invention may be that hydrogen gas may be produced in a relatively effective manner. Particularly, by using microbial electrolysis cell processing rather than conventional electrolysis, the voltage applied for hydrogen gas generation may be lowered. In this respect it is noted that the voltage may be reduced both below the typical optimum voltage levels of conventional electrolysis facilities, and in many cases even below the theoretical minimum voltage levels necessary for conventional hydrogen electrolysis.

A further advantage of the invention may be that a more efficient processing may be obtained. When processing the first biomass by means of the microbial electrolysis cell processing, the retention time of the biomass may be significantly reduced when compared to a conventional processing by anaerobic digestion. This results in a processing system which may be significantly more efficient.

A further advantage of the invention may be that by employing processing in a microbial electrolysis cell reactor of the first biomass, a higher quality gas may be obtained. For example, when producing methane in the microbial electrolysis cell reactor, the concentration of methane in the output gas from the microbial electrolysis cell reactor may be significantly higher than the methane concentration in the output of anaerobic digestion. This may in turn be advantageous as cleanup of the gas e.g. by CO2 removal may be costly.

In the present context the term “first inlet” refers to the any suitable receiver for receiving the first biomass. In some embodiments it may be an inlet for connecting to a tank truck, a train tank car, or similar. In some embodiments, the inlet may represent the coupling to e.g. a piping system for receiving the first biomass. In some embodiments, it may be an inlet from a storage container.

In the present context the term “second inlet” refers to the any suitable receiver for receiving the second biomass. In some embodiments it may be an inlet for connecting to a tank truck, a train tank car, or similar. In some embodiments, the inlet may represent the coupling to e.g. a piping system for receiving the second biomass. In some embodiments, it may be an inlet from a storage container.

In the present context the term “anaerobic digestion reactor” refers to a reactor for performing anaerobic digestion, i.e. breakdown of biomass by microbes at oxygen deficient conditions, i.e. at zero or very low content of oxygen. In the present context, anaerobic microorganisms facilitate the breakdown of biomass, eventually resulting in biogas containing methane and carbon dioxide as its main constituents. Such microorganisms may e.g. comprise hydrolysis performing microorganisms, acidogenic microorganisms, acetogenic microorganisms, methanogenic microorganisms etc. It is noted that the terms “anaerobic digestion reactor”, “anaerobic digester”, and “anaerobic reactor” may be used interchangeably.

In the present context the term “microbial electrolysis cell reactor” refers to a reactor for processing by electrogenic microorganisms consuming a biomass and its degradation products to produce methane or hydrogen gas when an external voltage is applied. More specific, the terms “microbial electrolysis cell” and “microbial electrolysis cell reactor” is used interchangeably to refer to the device configured for performing such process.

In the present context the term “first output” refers to any suitable outlet for discharging the first biomass digestate. In some embodiments it may be an outlet for connecting to a tank truck, a train tank car, or similar. In some embodiments, the inlet may represent the coupling to e.g. a piping system for receiving the first biomass digestate. In some embodiments, it may be an outlet into a storage container.

In the present context the term “second output” refers to any suitable outlet for discharging the second biomass digestate. In some embodiments it may be an outlet for connecting to a tank truck, a train tank car, or similar. In some embodiments, the inlet may represent the coupling to e.g. a piping system for receiving the second biomass digestate. In some embodiments, it may be an outlet into a storage container.

In the present context the term “biogas” intended to mean a product gas comprising methane gas. Biogas is obtained from degradation of biological material, such as biomass. Typically, raw or unprocessed biogas comprises methane gas and carbon dioxide gas as its main constituents, whereas processed biogas is composed of methane or at least mainly of methane. Minor amounts of e.g. hydrogen sulfide and water vapor may also be present. Typically, the raw biogas may be upgraded or purified to increase the relative content of methane, e.g. in view of legal limits on minimum methane content and/or maximum content of certain other gasses. The content of other constituents in processed biogas may vary, e.g. due to legal requirements and also based on the specific composition of the used biomass. In some embodiments, raw or unprocessed biogas comprises methane in an amount of at least 40% by volume of the biogas, such as 40-90% by volume of the biogas. In some embodiments, the processed biogas comprises methane in an amount of at least 95% by volume of the biogas, such as 95-99.99% by volume of the biogas.

Additionally, when referring to “methane”, this may be in the form of biogas, either produced by the anaerobic digestion reactor or by the microbial electrolysis cell reactor, or may be produced by a biogas upgrader by using carbon dioxide from the anaerobic digestion reactor and hydrogen from the microbial electrolysis cell reactor in a methanation reaction to produce methane.

As used herein, the term “dry matter” is intended to mean the residual when water is evaporated.

As used herein, the term volatile solids (VS) shall mean the organic part of dry matter. Usually this is measured by heating a sample (which has been dried at 105 degrees Celsius) to e.g. 550 degrees Celsius, so that only salts and ashes remain.

In the context of fibrous biomass, the term “particle size” is understood as a longest dimension of the particles in the biomass. Therefore, fibrous biomass having a particle size of at least 1 cm refers to fibrous biomass of particles having a length of at least 1 cm in at least one dimension. This may be measured e.g. by using a sieving tower.

In an advantageous embodiment of the invention, the microbial electrolysis cell reactor is configured for producing hydrogen.

In an advantageous embodiment of the invention, the microbial electrolysis cell reactor is configured for producing biogas.

In an advantageous embodiment of the invention, the microbial electrolysis cell reactor is a two-chamber microbial electrolysis cell comprising a membrane.

By including a membrane, the microbial electrolysis cell produces hydrogen gas. It is noted that some methane may also be produced as a biproduct when the microbial electrolysis cell produces hydrogen gas. Carbon dioxide is also produced as from the anodic compartment. Other minor gas components may be produced.

In an advantageous embodiment of the invention, the microbial electrolysis cell reactor is a one-chamber microbial electrolysis cell.

By omitting a membrane, the microbial electrolysis cell produces methane. Additionally, carbon dioxide will be produced, although substantial amounts of this may be converted to methane. Other minor gas components may be produced.

In an advantageous embodiment of the invention, the system is further configured for initializing the microbial electrolysis cell reactor by injecting an initializing biomass comprising filtered second biomass digestate.

The initializing step may also be referred to as inoculation.

In an advantageous embodiment of the invention, the system further comprises a biogas upgrader for reducing the content of carbon dioxide in the biogas.

In an embodiment of the invention, the biogas upgrader comprises one or more purification steps based on water scrubbing, pressure swing adsorption, solvent adsorption, membrane filtration, and amine gas treating.

Solvent adsorption may e.g. be based on dimethyl ether of polyethylene glycol, such known as the Selexol process.

Especially in embodiments where the product gas consists of biogas, the biogas obtained from the anaerobic digestion may be subjected to methanization, such as biomethanization. This may comprise using hydrogen produced in the microbial electrolysis cell reactor for converting carbon dioxide in the biogas to methane.

In an advantageous embodiment of the invention, the biogas upgrader is connected to microbial electrolysis cell reactor for receiving biogas and/or hydrogen gas and to the anaerobic digestion reactor for receiving biogas.

In an embodiment of the invention, the biogas upgrader is connected to microbial electrolysis cell reactor for receiving biogas and/or hydrogen gas.

In an embodiment of the invention, the biogas upgrader is connected to the anaerobic digestion reactor for receiving biogas.

In an advantageous embodiment of the invention, the biogas upgrader is further configured for reducing the content of at least one of hydrogen sulfide, water, and carbon monoxide.

In an embodiment of the invention the system further comprises a methanation unit configured to convert carbon dioxide from the anaerobic digestion reactor to methane by methanation.

In an advantageous embodiment of the invention, the system further comprises a methanation unit configured to convert carbon dioxide from the anaerobic digestion reactor to methane by methanation using hydrogen received from the microbial electrolysis cell reactor.

In an advantageous embodiment of the invention, the system is configured to dilute the first biomass (FBM) before the microbial electrolysis cell reactor (MECR).

Thus, in the above embodiment, the first biomass is diluted at the point of adding it to the microbial electrolysis cell reactor.

In an advantageous embodiment of the invention, the system is configured to dilute the first biomass (FBM) to a COD of at least 10,000 mg O2/L before the microbial electrolysis cell reactor (MECR), such as at least 20,000 mg O2/L before the microbial electrolysis cell reactor (MECR), such as at least 50,000 mg O2/L, such as at least 75,000 mg O2/L.

In an embodiment of the invention, the system is configured to dilute the first biomass (FBM) to a COD of 10,000-1,000,000 mg O2/L before the microbial electrolysis cell reactor (MECR), such as 20,000-1,000,000 mg O2/L before the microbial electrolysis cell reactor (MECR), such as 50,000-1,000,000 mg (2/L, such as 75,000-1,000,000 mg O2/L.

In an advantageous embodiment of the invention, the system is configured to dilute the first biomass (FBM) to a content of nitrogen of at least 2 gram per kg before the microbial electrolysis cell reactor (MECR), such as at least 3 gram per kg.

In an embodiment of the invention, the first biomass has a content of nitrogen of 2-50 gram per kg, such as 3-50 gram per kg.

In an advantageous embodiment of the invention, the system is configured to dilute the first biomass (FBM) to a content of phosphorous of at least 3 gram per kg before the microbial electrolysis cell reactor (MECR), such as at least 4 gram per kg.

In an embodiment of the invention, the first biomass has a content of phosphorous of 3-50 gram per kg, such as 4-50 gram per kg.

In an advantageous embodiment of the invention, the system is configured to dilute the first biomass (FBM) to a content of potassium of at least 3 gram per kg before the microbial electrolysis cell reactor (MECR), such as at least 4 gram per kg.

In an embodiment of the invention, the first biomass has a content of potassium of 3-50 gram per kg, such as 4-50 gram per kg.

In an embodiment of the invention, the system is configured to dilute the first biomass (FBM) before the microbial electrolysis cell reactor (MECR), and wherein the first biomass in diluted form has a chemical oxygen demand (COD) of at least 10,000 mg O2/L and also at least one of
a content of nitrogen of at least 2 gram per kg,
a content of phosphorous of at least 3 gram per kg, and
a content of potassium of at least 3 gram per kg.

In an advantageous embodiment of the invention, the first biomass (FBM) introduced to the microbial electrolysis cell reactor (MECR) in undiluted form.

Thus, in the above embodiment, the first biomass is introduced to the microbial electrolysis cell reactor in undiluted form having a chemical oxygen demand (COD) of at least 100,000 mg O2/L while also having at least one of
a content of nitrogen of at least 4 gram per kg,
a content of phosphorous of at least 5 gram per kg, and
a content of potassium of at least 5 gram per kg.

In an advantageous embodiment of the invention, the system further comprises an error state monitoring system.

In an advantageous embodiment of the invention, the error state monitoring system comprises measuring one or more parameters selected from dry matter content change, pH, temperature, biogas composition, and volatile fatty acid content in the digestate.

In an embodiment of the invention, the biogas composition, and volatile fatty acid content in the digestate may be measured by means of gas chromatography and/or high-performance liquid chromatography.

In an advantageous embodiment of the invention, the error state monitoring system is connected to measure on the anaerobic digestion reactor and/or the microbial electrolysis cell reactor.

In an advantageous embodiment of the invention, the system further comprises a separator for separating a solid fraction from the first biomass.

I.e. the step of separating a solid fraction is prior to the step of processing the first biomass in a microbial electrolysis cell reactor. In a corresponding method for producing biogas from biomasses, the method further comprises a step of separating a solid fraction from the first biomass.

Especially when including dairy waste in the first biomass, it may be advantageous to separate a fraction from the dairy waste, as such fractions may be undesirable, e.g. by leading to clogging in the microbial electrolysis cell reactor or due to low processability of such fraction.

In an advantageous embodiment of the invention, the system further comprises a liquid input arranged to add liquid to the first biomass.

The liquid input is connected to the first biomass before the microbial electrolysis cell reactor or is separately connected to the microbial electrolysis cell reactor.

In embodiments where the system comprises a separator arranged to separate a solid fraction from the first biomass, the liquid input may advantageously be connected after this separation.

In an advantageous embodiment of the invention, the liquid input comprises a backmix feeding line configured to mix a part of the first biomass digestate with the first biomass.

Thus, in the above embodiment, some of the first biomass digestate, i.e. after processing in the microbial electrolysis cell reactor, is mixed into the first biomass, i.e. before the processing in the microbial electrolysis cell reactor. The backmix feeding line may also be referred to as a feedback dilution loop.

In a corresponding method for producing biogas from biomasses, the method further comprises a backmix step where a part of the first biomass digestate is mixed into the first biomass.

In an embodiment of the invention, the system comprises a backmix feeding line configured to mix a part of the first biomass digestate with the first biomass.

In an embodiment of the invention, the liquid input consists of a backmix feeding line configured to mix a part of the first biomass digestate with the first biomass.

In an advantageous embodiment of the invention, the backmix feeding line comprises a nitrogen removal unit arranged to lower the content of nitrogen in the backmix feeding line.

In an embodiment of the invention, the nitrogen removal unit is configured to increase the temperature and/or reduce the pressure of the backmix feeding line so as to remove nitrogen and optionally also reduce the salt content. Removing nitrogen and optionally also salt may be especially advantageous when the first biomass comprises molasses.

In an advantageous embodiment of the invention, the liquid input comprises nutrients such as macronutrients and/or micronutrients.

In an advantageous embodiment of the invention, the liquid input comprises a liquid second digestate fraction.

In the present context it is understood that the liquid second digestate fraction is obtained as the liquid fraction of a solid-liquid separation of the second digestate fraction.

In an advantageous embodiment of the invention, the microbial electrolysis cell reactor has a capacity of at least 5 m3, such as at least 20 m3, such as at least 50m3.

In an embodiment of the invention, the microbial electrolysis cell reactor has a capacity of 5-1,000 m3, such as 20-500 m3, such as 50-200 m3.

In some embodiments, two or more microbial electrolysis cell reactors may be used, e.g. in parallel, each of which may have the above defined capacity.

In one embodiment, the two or more microbial electrolysis cell reactors having a total capacity of 500-4,000 m3 are used.

In an advantageous embodiment of the invention, the anaerobic digestion reactor has a capacity of at least 500 m3, such as at least 1,000 m3, such as at least 2,000 m3.

In an embodiment of the invention, the anaerobic digestion reactor has a capacity of 500-20,000 m3, such as 1,000-10,000 m3, such as 2,000-5,000 m3.

In an advantageous embodiment of the invention, the system further comprises a first input storage container configured to receive said first biomass from the first inlet.

In such system, the first input storage container may be connected to the microbial electrolysis cell reactor and adapted to feed said first biomass to said microbial electrolysis cell reactor, directly or indirectly e.g. via intermediate processing.

In a corresponding method for producing biogas from biomasses, the method further comprises a step of storing, in a first input storage container, said first biomass.

In an advantageous embodiment of the invention, the first input storage container has a capacity of at least 30 m3, such as at least 50 m3, such as at least 100 m3.

In an embodiment of the invention, the first input storage container has a capacity of 30-10,000 m3, such as 50-5,000 m3, such as 100-2,000 m3.

In an advantageous embodiment of the invention, the system further comprises a second input storage container configured to receive said second biomass from the second inlet.

In such system, the second input storage container may be connected to an anaerobic reactor and adapted to feed said second biomass to said anaerobic reactor, directly or indirectly e.g. via intermediate processing.

In a corresponding method for producing biogas from biomasses, the method further comprises a step of storing, in a second input storage container, said second biomass.

In an advantageous embodiment of the invention, the second input storage container has a capacity of at least 30 m3, such as at least 50 m3, such as at least 100 m3.

In an embodiment of the invention, the second input storage container has a capacity of 30-10,000 m3, such as 50-5,000 m3, such as 100-2,000 m3.

In an advantageous embodiment of the invention, the system further comprises a first output storage container configured to receive said first biomass, the first output storage being connected to the first output.

Thus, the first output storage container is connected to the microbial electrolysis cell reactor, either directly from the microbial electrolysis cell reactor or after additional processing, and adapted to receive said first biomass after processing in said microbial electrolysis cell reactor.

In a corresponding method for producing biogas from biomasses, the method further comprises a step of storing, in a first output storage container, said first biomass digestate. Thus, a step of discharging the first biomass digestate would comprise discharging the first biomass digestate from the first output storage container.

In an advantageous embodiment of the invention, the system further comprises a second output storage container configured to receive said second biomass digestate, the second output storage being connected to the second output.

The second output storage container is connected to the anaerobic digestion reactor, either directly from the anaerobic digestion reactor or after additional processing, and adapted to receive said second biomass after processing in said anaerobic digestion reactor.

In a corresponding method for producing biogas from biomasses, the method further comprises a step of storing, in a second output storage container, said second biomass digestate. Thus, a step of discharging the second biomass digestate would comprise discharging the second biomass digestate from the second output storage container.

In an advantageous embodiment of the invention, the first biomass comprises one or more selected from dairy waste, molasse, fish waste, and food waste.

In the present context, food waste may refer to food-based waste, such as both waste resulting from food production and also consumer generated food waste.

In an advantageous embodiment of the invention, the first biomass further comprises one or more selected from food oils and fats, and glycerin.

In an advantageous embodiment of the invention, the first biomass comprises dairy waste and/or molasse.

In an embodiment of the invention, the first biomass consists of dairy waste and/or molasse.

In an embodiment of the invention, the first biomass comprises dairy waste.

In an embodiment of the invention, the first biomass comprises molasse.

In an embodiment of the invention, the first biomass consists of dairy waste.

In an embodiment of the invention, the first biomass consists of molasse.

In an advantageous embodiment of the invention, the first biomass has a chemical oxygen demand (COD) of 100,000-1,000,000 mg O2/L.

In an embodiment of the invention, the first biomass has a chemical oxygen demand

(COD) of at least 150,000 mg O2/L, such as at least 200,000 mg O2/L.

In an embodiment of the invention, the first biomass has a chemical oxygen demand (COD) of 150,000-1,000,000 mg O2/L, such as 150,000-1,000,000 mg O2/L.

Thus, in the above embodiment the first biomass has a chemical oxygen demand (COD) in the range of 105-106 mg O2/L

In an advantageous embodiment of the invention, the first biomass has a dry matter content of 15-90% by weight of the first biomass.

In an embodiment of the invention, the first biomass has a dry matter content of at least 20% by weight of the first biomass, such as at least 25% by weight of the first biomass.

In an embodiment of the invention, the first biomass has a dry matter content of 20-85% by weight of the first biomass, such as 25-80% by weight of the first biomass.

In an advantageous embodiment of the invention, the first biomass has a content of nitrogen of 4-50 gram per kg.

In an advantageous embodiment of the invention, the first biomass has a content of phosphorous of 5-50 gram per kg.

In an advantageous embodiment of the invention, the first biomass has a content of potassium of 5-50 gram per kg.

In an advantageous embodiment of the invention, the first biomass is free of wastewater.

In an advantageous embodiment of the invention, the second biomass has a content of nitrogen, phosphorous, and potassium, each being less than 5 gram per kg.

In an embodiment of the invention, the second biomass has a content of nitrogen, phosphorous, and potassium, each being 0.01-5 gram per kg, such as 0.1-5 gram per kg.

In an embodiment of the invention, the second biomass has a content of nitrogen being less than 4 gram per kg.

In an embodiment of the invention, the second biomass has a content of nitrogen being 0.01-4 gram per kg, such as 0.1-4 gram per kg.

In an embodiment of the invention, the second biomass has a content of phosphorous being less than 5 gram per kg.

In an embodiment of the invention, the second biomass has a content of phosphorous being 0.01-5 gram per kg, such as 0.1-5 gram per kg.

In an embodiment of the invention, the second biomass has a content of potassium being less than 5 gram per kg.

In an embodiment of the invention, the second biomass has a content of potassium being 0.01-5 gram per kg, such as 0.1-5 gram per kg.

In an advantageous embodiment of the invention, the second biomass has a chemical oxygen demand (COD) of at least 5,000 mg O2/L, such as at least 10,000 mg O2/L.

In an embodiment of the invention, the second biomass has a chemical oxygen demand (COD) of 5,000-100,000 mg O2/L, such as 10,000-50,000 mg O2/L.

In an embodiment of the invention, the second biomass has a COD content of at least 20,000 mg O2/L, such as at least 40,000 mg (2/L, such as at least 60,000 mg O2/L.

In an embodiment of the invention, the second biomass has a COD content of 20,000-200,000 mg O2/L, such as 40,000-150,000 mg O2/L, such as 60,000-100,000 mg O2/L.

In an embodiment of the invention, the first biomass has COD that is at least 50,000 mg O2/L higher than the second biomass, such as at least 100,000 mg O2/L.

In an advantageous embodiment of the invention, the second biomass comprises straw.

In an advantageous embodiment of the invention, the second biomass has a dry matter content of at least 10% by weight of the second biomass, such as at least 15% by weight of the second biomass.

In an embodiment of the invention, the second biomass has a dry matter content of 10-90% by weight of the second biomass, such as 15-90% by weight of the second biomass.

According to an embodiment of the invention, the second biomass comprises farm-based components, such as animal feces containing fractions and/or crop fractions.

Animal feces fractions may for example include manure and/or deep litter. Crop fractions may e.g. include aftercrop components.

According to an embodiment of the invention, the second biomass comprises fibrous biomass, such as fibrous biomass having a particle size of at least 1 cm.

In an embodiment of the invention, the second biomass comprises fibrous biomass, such as fibrous biomass having a particle size of 1-15 cm.

According to an embodiment of the invention, the second biomass comprises fibrous biomass having a particle size of at least 1 cm in an amount of at least 2% by weight of the second biomass, such as at least 5% by weight of the second biomass, such as at least 10% by weight of the second biomass.

In an embodiment of the invention, the second biomass comprises fibrous biomass having a particle size of at least 1 cm in an amount of 2-40% by weight of the second biomass, such as 5-30% by weight of the second biomass, such as 10-20% by weight of the second biomass.

In an embodiment of the invention, the second biomass comprises fibrous biomass having a particle size of 1-15 cm in an amount of 2-40% by weight of the second biomass, such as 5-30% by weight of the second biomass, such as 10-20% by weight of the second biomass.

Typical fibrous biomasses and fibrous biomasses containing fractions in the above context may include straw, deep litter, hay, corn straw, grass, and any combination thereof.

In an embodiment of the invention, biochar is added to the first biomass. Biochar is well-known and may be produced in a number of different ways, for example by means of pyrolysis. In the context of the present invention, the pyrolysis may even use the second biomass digestate or a fraction thereof as input. The addition of biochar may advantageously increase the efficiency of the microbial electrolysis cell processing.

In an advantageous embodiment of the invention, the system further comprises a second microbial electrolysis cell reactor configured to receive the second biomass after processing in said anaerobic digestion reactor, and to feed the second biomass to the second output after processing.

Thus, in the above embodiment, the second biomass is feed to the second microbial electrolysis cell reactor after processing in said anaerobic digestion reactor. This may be directly or after intermediate processing. After the second biomass is processed in the second microbial electrolysis cell reactor, the resulting second biomass digestate is feed to the second output, directly or after intermediate processing. In the above embodiment, where the system comprises a second microbial electrolysis cell reactor, the microbial electrolysis cell reactor for processing the first biomass may also be referred to as the first microbial electrolysis cell reactor.

In a corresponding method for producing biogas from biomasses, the method further comprises the step of processing the second biomass digestate in a microbial electrolysis cell reactor to produce biogas and/or hydrogen.

In an advantageous embodiment of the invention, the second microbial electrolysis cell reactor and the microbial electrolysis cell reactor for processing the first biomass are separate units.

In an advantageous embodiment of the invention, the system further comprises a first inlet pump configured to feed the first biomass from the first inlet to the microbial electrolysis cell reactor.

In an advantageous embodiment of the invention, the first inlet pump has a pumping capacity of at least 6 m3 per hour, such as at least 10 m3 per hour, such as at least 15 m3 per hour.

In an embodiment of the invention, the first inlet pump has a pumping capacity of 6-100 m3 per hour, such as 10-80 m3 per hour, such as 15-60 m3 per hour.

In an advantageous embodiment of the invention, the system further comprises a second inlet pump configured to feed the second biomass from the second inlet to the anaerobic digestion reactor.

In an advantageous embodiment of the invention, the second inlet pump has a pumping capacity of at least 6 m3 per hour, such as at least 10 m3 per hour, such as at least 15 m3 per hour.

In an embodiment of the invention, the second inlet pump has a pumping capacity of 6-100 m3 per hour, such as 10-80 m3 per hour, such as 15-60 m3 per hour.

In an advantageous embodiment of the invention, the system further comprises a first output pump configured to feed the first biomass digestate from the microbial electrolysis cell reactor to the first output.

In an advantageous embodiment of the invention, the first output pump has a pumping capacity of at least 0.2 m3 per hour, such as at least 0.5 m3 per hour, such as at least 1 m3 per hour.

In an embodiment of the invention, the first output pump has a pumping capacity of 0.2-34 m3 per hour, such as 0.5-30 m3 per hour, such as 1-20 m3 per hour.

In an advantageous embodiment of the invention, the system further comprises a second output pump configured to feed the second biomass digestate from the anaerobic digestion reactor to the second output.

In an advantageous embodiment of the invention, the second output pump has a pumping capacity of at least 0.2 m3 per hour, such as at least 0.5 m3 per hour, such as at least 1 m3 per hour.

In an embodiment of the invention, the second output pump has a pumping capacity of 0.2-34 m3 per hour, such as 0.5-30 m3 per hour, such as 1-20 m3 per hour.

In an advantageous embodiment of the invention, the first outlet and the second outlet are controllable to discharge a predefined amount of the first biomass digestate and the second biomass digestate.

Thereby, the total discharged amount of digestate may have a ratio between certain constituents, in particular nutrients including phosphorous and nitrogenous compounds, which is adjustable by means of the first outlet and the second outlet.

The invention further relates to a system for producing product gas comprising methane, the system comprising
a first inlet for receiving a first biomass,
a second inlet for receiving a second biomass,
a microbial electrolysis cell reactor for processing the first biomass to produce methane and/or hydrogen gas and a first biomass digestate,
an anaerobic digestion reactor for processing the second biomass to produce methane and a second biomass digestate,
a first output for discharging the first biomass digestate,
a second output for discharging the second biomass digestate.

The invention further relates to a method of producing biogas from biomasses, the method comprising the steps of
providing a first biomass, the first biomass (FBM) having a dry matter content of at least 15% by weight of the first biomass (FBM) and a chemical oxygen demand (COD) of at least 100,000 mg O2/L, the first biomass (FBM) having at least one of
   a content of nitrogen of at least 4 gram per kg,
   a content of phosphorous of at least 5 gram per kg, and
   a content of potassium of at least 5 gram per kg.
a second inlet (SIN) for receiving a second biomass (SBM),
providing a second biomass,
processing the first biomass, optionally in diluted form, in a microbial electrolysis cell reactor to produce biogas and/or hydrogen and a first biomass digestate,
subjecting the second biomass to anaerobic digestion processing to produce biogas and a second biomass digestate,
discharging the first biomass digestate, discharging the second biomass digestate.

In an embodiment of the invention, the first biomass comprises dairy waste.

In an embodiment of the invention, the first biomass comprises molasse.

In an embodiment of the invention, the first biomass is selected from dairy waste and/or molasse.

In an embodiment of the invention, the method is carried out using a system according to the invention or any of its embodiments.

The invention further relates to a method of producing biogas from biomasses, the method comprising the steps of
providing a first biomass selected from dairy waste and/or molasse,
providing a second biomass,
processing the first biomass in a microbial electrolysis cell reactor to produce biogas and/or hydrogen and a first biomass digestate,
subjecting the second biomass to anaerobic digestion processing to produce biogas and a second biomass digestate,
discharging the first biomass digestate,
discharging the second biomass digestate.

In an advantageous embodiment of the invention, the method is carried out using a system according to the invention or any of its embodiments.

### FIGURES

The invention will now be described with reference to the figures, where

FIG. 1 illustrates a system AMS for producing product gas PG comprising methane according to an embodiment of the invention,

FIG. 2 illustrates a system AMS for producing product gas PG comprising methane according to an embodiment of the invention,

FIGS. 3A and 3B illustrate a microbial electrolysis cell reactor MECR according to embodiments of the invention,

FIGS. 4A and 4B illustrates a part of a system AMS for producing product gas PG comprising methane according to embodiments of the invention,

FIG. 5 illustrates an anaerobic digestion reactor ADR according to an embodiment of the invention,

FIG. 6 illustrates methane and hydrogen production of example 4,

FIG. 7 illustrates methane and hydrogen production of example 5,

FIG. 8 illustrates methane production of example 6, and

FIGS. 9a-9b illustrate methane production of examples 7a-7b.

### DETAILED DESCRIPTION

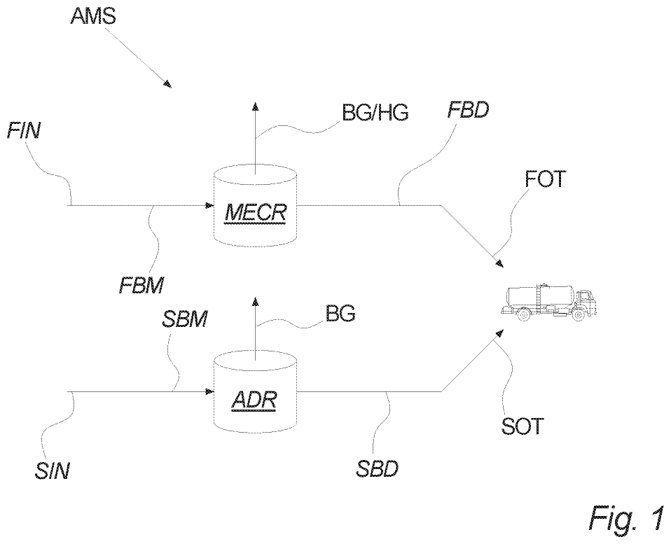

Referring to FIG. 1, a system AMS for producing product gas PG comprising methane according to an embodiment of the invention is illustrated.

As illustrated in FIG. 1, the system AMS comprises a first inlet FIN for receiving a first biomass FBM, a second inlet SIN for receiving a second biomass SBM, a microbial electrolysis cell reactor MECR, an anaerobic digestion reactor ADR, a first output FOT, and a second output SOT.

The first biomass has a dry matter content of at least 15% by weight of the first biomass FBM, a chemical oxygen demand (COD) of at least 100,000 mg O2/L. Additionally, the first biomass FBM has at least one of
a content of nitrogen of at least 4 gram per kg,
a content of phosphorous of at least 5 gram per kg, and
a content of potassium of at least 5 gram per kg.

The first inlet FIN is connected to the microbial electrolysis cell reactor MECR so as to feed the first biomass FBM into the microbial electrolysis cell reactor MECR. In some embodiments, the first biomass FBM is fed directly into the microbial electrolysis cell reactor MECR. In some embodiments, the first biomass FBM is pre-processed before being fed directly into the microbial electrolysis cell reactor MECR, e.g. by one or more of pre-treatment, addition of additives, dilution, and separation.

In the microbial electrolysis cell reactor MECR, the first biomass FBM is processed by microbial electrolysis cell processing to produce methane and/or hydrogen and a second biomass digestate SBD. Typically, the microbial electrolysis cell reactor MECR may be configured to produce biogas BG having methane as a main constituent or to produce hydrogen gas HG having carbon dioxide as a byproduct.

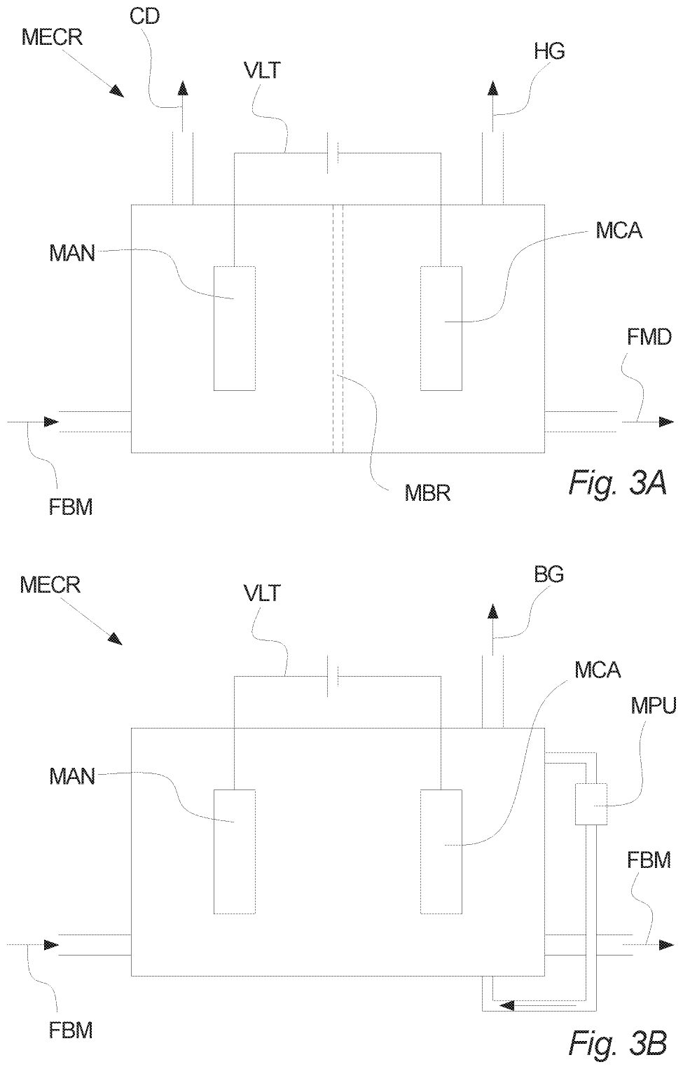

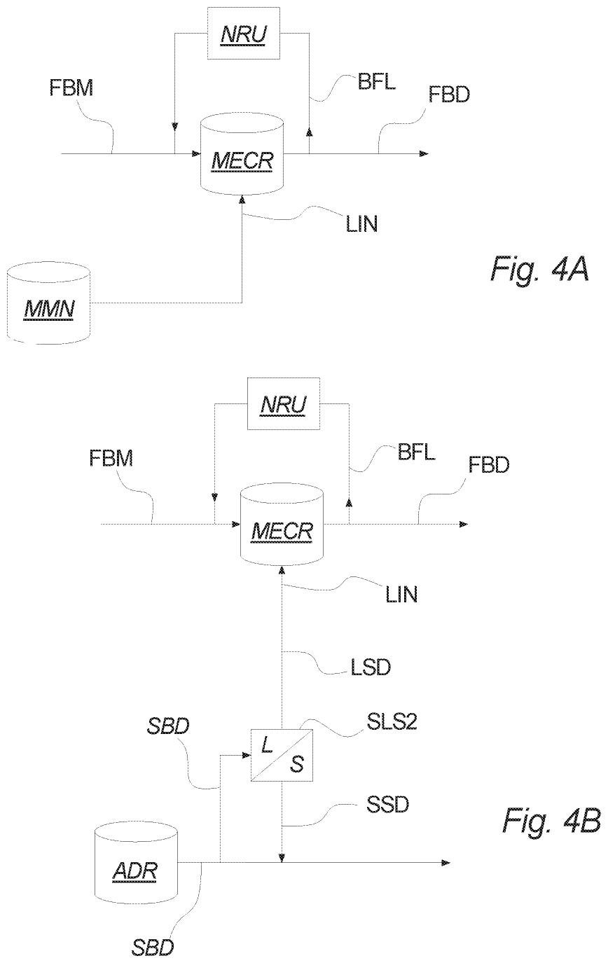

The microbial electrolysis cell reactors MECR shown FIGS. 3A and 3B may be used. Also, the aspects shown in FIGS. 4A-4B may be used in the context of FIG. 1.

The first biomass digestate FBD is then fed to the first output FOT, from which it can be discharged at a suitable time. In some embodiments the first biomass digestate FBD is fed directly to the first output FOT and discharged. In some embodiments, the first biomass digestate FBD is post-processed after the microbial electrolysis cell reactor MECR. In some embodiments the first biomass digestate FBD is fed to a storage before being discharged at a later time.

The second inlet SIN is connected to the anaerobic digestion reactor ADR so as to feed the second biomass SBM into the anaerobic digestion reactor ADR. In some embodiments, the second biomass SBM is fed directly into the anaerobic digestion reactor ADR. In some embodiments the second biomass SBM is pre-processed before being fed directly into the anaerobic digestion reactor ADR e.g. by one or more of pre-treatment, addition of additives, dilution, and separation.

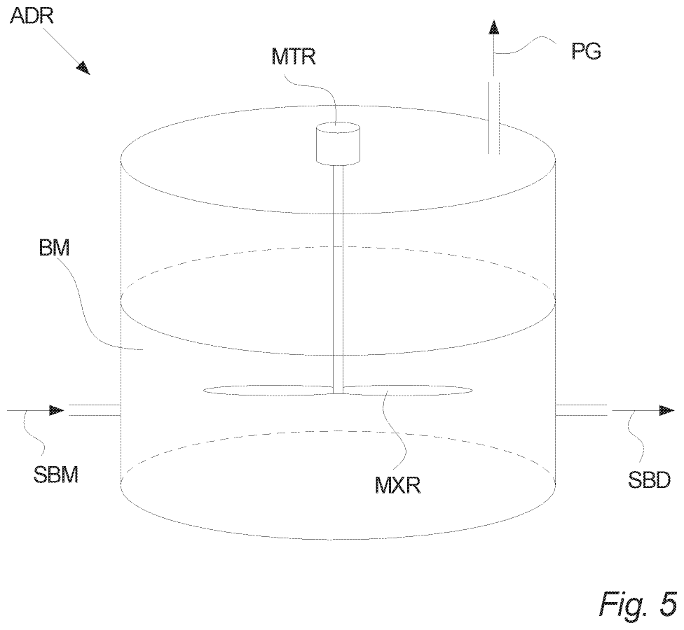

In the anaerobic digestion reactor ADR, the second biomass SBM is processed by anaerobic digestion to produce biogas BG having methane as a main constituent and a second biomass digestate SBD. The anaerobic digestion reactor shown in FIG. 5 may be used.

The second biomass digestate SBD is then fed to the second output SOT, from which it can be discharged at a suitable time. In some embodiments the second biomass digestate SBD is fed directly to the second output SOT and discharged. In some embodiments, the second biomass digestate SBD is post-processed after the anaerobic digestion reactor ADR. In some embodiments the second biomass digestate SBD is fed to a storage before being discharged at a later time.

As shown in FIG. 1, the first biomass digestate FBM and the second biomass digestate SBD may be discharged to a tank truck for transportation to a remote location. It is noted that the first biomass digestate FBM and the second biomass digestate SBD may be fed into the same tank truck and may thus be mixed therein, or may be fed into separate tank trucks and may thus be mixed subsequently. Also, it is noted that the first biomass digestate FBM and the second biomass digestate SBD may be discharged to other means of transportation, including but not limited to train tank cars, chips, pipelines, etc.

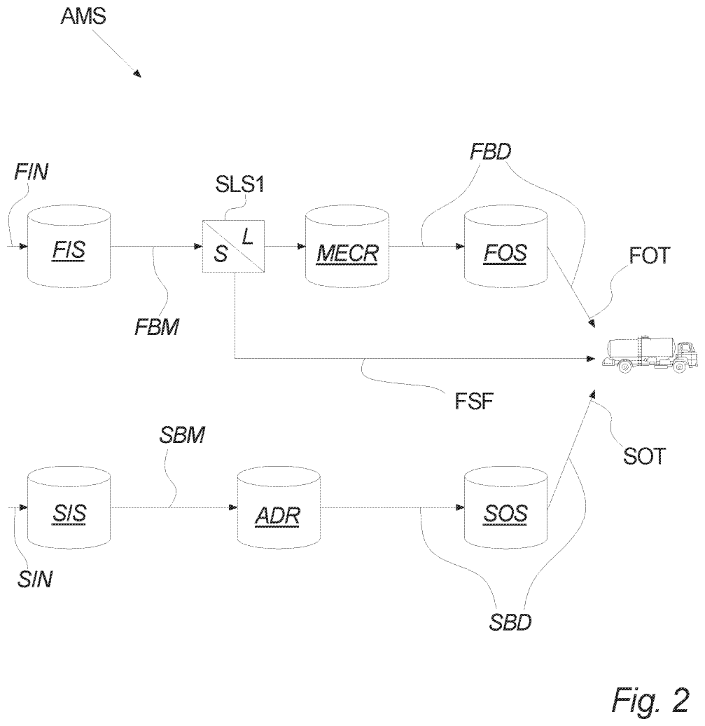

Now turning to FIG. 2, a system AMS for producing product gas PG comprising methane according to an embodiment of the invention is illustrated.

Further to the embodiment illustrated in FIG. 1, FIG. 2 illustrates that the first biomass FBM may be fed from the first inlet FIN into a first input storage FIS before being fed forward for processing in the microbial electrolysis cell reactor MECR.

Similarly, the second biomass SBM may be fed from the second inlet SIN into a second input storage SIS before being fed forward for processing in the anaerobic digestion reactor ADR.

Also, after being ejected from the microbial electrolysis cell reactor MECR, the first biomass digestate FBM is stored in a first output storage FOS before being discharged from the first output FOT.

Similarly, after being ejected from the anaerobic digestion reactor ADR, the second biomass digestate SBM is stored in a second output storage SOS before being discharged from the second output SOT.

Another aspect that is illustrated in FIG. 2, is the presence of a first solid liquid separator SLS1. The first solid liquid separator SLS1 is arranged to receive the first biomass FBM and separate a first solid fraction FSF therefrom.

The first solid fraction FSF may be fed directly to be discharged, e.g. in a similar manner as the first and second biomass digestates FBM, SBM. Thus, in some embodiments, the system may in addition to the first output storage FOS and/or the second output storage SOS comprise a storage (not shown) for storing the first solid fraction FSF before discharging thereof.

Additionally, it is noted that the aspect of having one or more input storage(s) and/or output storage(s) may be present independently from whether the first solid liquid separator SLS1 is included in the system AMS.

Referring to FIGS. 3A and 3B, microbial electrolysis cell reactors MECR according to embodiments of the invention are described.

The microbial electrolysis cell reactor MECR comprises an anode MAN and a cathode MCA, which in the embodiment of FIG. 3A is separated by a membrane MBR.

The anode MAN may comprise or be made from a number of different materials, including but not limited to carbon (e.g. in the form of carbon cloth, carbon paper, carbon felt, carbon foam, biochar, glassy carbon, carbon nanotube sponges, etc.), graphite (e.g. in the form of graphite felt, graphite granules, graphite brushes), conductive polymer-based composite material (e.g. using polymers such as polyaniline, polypyrrole, polythiophene, poly-co-o-aminophenol, etc.), metals and metal oxides, graphene derivatives with metal/metal oxide nanoparticles or conductive polymer-based composite materials.

The cathode MCA may comprise or be made from a number of different materials, including but not limited to carbon-based materials, composites, metals and metal oxides. Generally, similar material as for the anode may be used. Conductive materials are used to make electrodes, such as platinum meshes, carbon felt, carbon fibre, and carbon cloth. Catalysts, such as platinum and titanium, may to enhance performance of the cathode.

The first biomass FBM is injected to the microbial electrolysis cell reactor MECR through a suitable inlet and collected as first biomass digestate FBM by a suitable outlet after processing. It is noted that the specific configuration with respect to inlets and outlets for the first biomass FBM and first biomass digestate FBD may differ between specific embodiments.

The microbial electrolysis cell reactor MECR may further comprise one or more outlets for collecting gas produced during the microbial electrolysis cell processing. In two-chamber embodiments, two outlets may typically be used, one for collecting carbon dioxide CD from the anode and another outlet for collecting hydrogen gas HG from the cathode.

The membrane MBR as illustrated in FIG. 3A is configured to allow passage of protons produced by microorganism present at the anode. The passing protons are reduced at the cathode MCA due to a sufficiently high voltage VLT being applied. Thereby, hydrogen gas is formed, which may then be collected.

Additionally, carbon dioxide is formed at the anode by the microorganisms in the same reaction as the protons. The carbon dioxide may then be collected besides the hydrogen gas as the two main constituents gasses produced by the microbial electrolysis cell MECR.

In the embodiment illustrated in FIG. 3B, no membrane is applied, and therefore carbon dioxide may therefore be present at the cathode, whereby microorganisms may produce methane and water from the hydrogen and carbon dioxide.

Consequently, a single outlet for collecting biogas BG may be used for single chamber embodiments.

Another aspect illustrated on FIG. 3B is that the microbial electrolysis cell reactor MECR further comprises a MEC pump MPU, which is arranged to pump biomass digestate BDM from one part of the microbial electrolysis cell reactor MECR to another part of the microbial electrolysis cell reactor MECR in a loop, whereby agitation in the biomass digestate BDM is introduced and any tendency for clogging may further be reduced. The pumping flow of the MEC pump MCU may be adjusted depending e.g. on the composition of the biomass digestate BDM. As an example, if the biomass digestate BDM having a higher tendency to introduce clogging is used, the pump flow may be increased to provide more agitation. It is noted that the use of a MEC pump MPU may be done independent on other aspects illustrated on FIG. 3B, and may also be usable with two-chamber MECs, such as the MEC illustrated in FIG. 3A.

It is noted that the design illustrated in FIGS. 3A and 3B is only a schematic representation, and that this may be implemented in a number of different ways. As an illustrative example, electrodes may e.g. comprise several plates in in certain forms, such as cylinder form.

It is noted that depending on the specific design, membrane used, etc. the produced gas may contain both hydrogen and methane, however, the embodiments in FIGS. 3A and 3B serves to illustrate how to influence the production in the direction of hydrogen and methane, respectively.

Referring to FIGS. 4A and 4B, a part of a system AMS for producing product gas PG comprising methane according to embodiments of the invention is illustrated.

The part of the system AMS shown in FIGS. 4A and 4B may each e.g. be implemented in the embodiment described with reference to FIG. 1.

As shown in both FIGS. 4A and 4B, a first biomass FBM is received at the microbial electrolysis cell reactor MECR. A part of the resulting first biomass digestate FBD is fed through a feedback dilution loop BFL, whereby the incoming first biomass FBM may be diluted without addition of external water. In some alternative embodiments, the dilution may be performed using external water or with a combination of the feedback dilution loop BFL and external water.

In anaerobic digestion, degradation of biomasses with high nitrogen fractions can lead to ammonia buildup which could inhibit the biogas production process. Most commercially applied nitrogen removal, in the form of ammonia, in anaerobic digestion rely on physico-chemical reactions. For instance, the nitrogen removal unit (NRU) could consist of an air or steam stripping unit coupled with gas washing in sulfuric acid to capture the ammonia. Alternatively, the NRU could house filling material such as for example zeolite, clay minerals, activated carbon, resins or functionalized surfaces, which can absorb the ammonium ion in an ion exchange or absorption process to lower nitrogen levels. Additionally, ultrasonic cavitation or microwave treatment are other viable NRU alternatives towards the removal of ammonia nitrogen. When using a feedback dilution loop BFL, a nitrogen removal unit NRU may be included to lower the nitrogen content of the part of the first biomass digestate that is used for dilution. This may prevent or lower any undesirable buildup of nitrogen due to the backmixing. In some embodiments, removal or lowering of certain undesirable salt may also be implemented.

Furthermore, in both FIGS. 4A and 4B, a liquid input LIN is shown. The liquid input LIN may be used to add certain nutrients to optimize the balance of macronutrients and/or micronutrients in the microbial electrolysis cell reactor MECR.

Referring specifically to the embodiment illustrated in FIG. 4A, a macronutrient and/or micronutrients storage MMN is used as a source of macronutrients, such as nitrogen, phosphorous, and potassium, and/or as a source of micronutrients such as minerals etc. It is noted that depending on the specific composition of the first biomass FBM and/or the microbial cultures present in the microbial electrolysis cell reactor MECR, the balance of the individual macronutrients and/or micronutrients may be adjusted.

Now, referring to the embodiment illustrated in FIG. 4B, a part of the second biomass digestate SBD is fed into a second solid liquid separator SLS2 arranged to separate a liquid second digestate fraction LSD from the second biomass digestate. Also resulting from the second solid liquid separator SLS2 is a solid second digestate fraction SSD, which may be returned to the remaining part of the second biomass digestate SBD.

Then, the liquid second digestate fraction LSD may be added via the liquid input LIN.

It is noted that the liquid input LIN of FIGS. 4A and 4B may alternatively be connected to the feeding line for the first biomass digestate FBM, i.e. before entering the microbial electrolysis cell reactor MECR.

In principle, the embodiments of FIGS. 4A and 4B may be combined. Thus, the liquid input LIN may also in some embodiments be fed both from the macronutrient and/or micronutrients storage MMN and from a liquid second digestate fraction LSD of the second biomass digestate SBD. Thereby, the capacity of the macronutrient and/or micronutrients storage MMN may be reduced.

The liquid input LIN may in some embodiments be understood as comprising the feedback dilution loop BFL.

Also, it is noted that the embodiments shown in FIG. 4A-4B are combinable with the embodiments of FIG. 2A-2B.

Referring to FIG. 5, an anaerobic digestion reactor ADR according to an embodiment of the invention is described.

As shown in FIG. 5, the second biomass SBM is received through a suitable biomass inlet. After anaerobic digestion, the resulting second biomass digestate SBD is ejected by a suitable outlet.

During the anaerobic digestion, the second biomass SBM may be agitated by a suitable mixer MXR brought into rotation by a motor MTR. Depending on the circumstances, such as the specific composition of the second biomass SBM, hereunder dry matter content, content of large particle sized fibrous biomass, etc., the rotational speed of the mixer MXR may be varied by the motor MTR.

### EXAMPLES

### Example 1—Example Method

Various biomasses may be fed into the MEC reactor.

As shown in the below table 1, total solids (corresponding to dry matter content) and volatile solids are shown for three biomasses FBM1-FMB3 used as first biomass. Also, a comparative biomass COM1 of wastewater is shown.

TABLE 1
Dry-and organic matter content of delactosed permeate, soya-and sugar
beet molasses. The dry matter content (TS) and organic content (VS) are
both based on fresh weight (FW) of the sample.

| | FBM1- | | | |
|---|---|---|---|---|
| | Delactosed | FBM2-Soya | FBM3-Sugar | COM1- |
| | permeate | molasse | beet molasse | Wastewater |
| TS [wt %] | 42.9 | 48.7 | 75.7 | 0.14 |
| VS [wt %] | 26.7 | 32.6 | 51.1 | 0.04 |

### Example 2—Simulation of Gas Volume from MEC of Wastewater, Delactosed Permeate and Molasses for Comparison of Outcome Based on Fresh Weight

Using simulations based on the VS of each sample, it is possible to compare the production of H₂ and CO₂ from each organic substance. In this example, a two chambered MEC is used. Therefore, the production of H₂ and CO₂ will come from a two chambered MEC, where CO₂ will be produced at the anode and H₂ at the cathode.

The simulation model was run with the following conditions:
Two-chamber MEC
An efficiency of the MECs conversion of the organic material of 72.5%
All VS content will be converted as if glucose or acetate

From here the calculations are as follow

$${H}_{2} production [ \frac{L}{{m}_{FW}^{3}} ] : {VS}_{subtrate} [ \% ] * 1000 \frac{\%}{\frac{g}{L}} * 1000 \frac{L}{{m}^{3}} * 1.49 \frac{{L}_{{H}_{2}}}{{g}_{Glucose}}$$

$${CO}_{2} production [ \frac{L}{{m}_{FW}^{3}} ] : {VS}_{subtrate} [ \% ] * 1000 \frac{\%}{\frac{g}{L}} * 1000 \frac{L}{{m}^{3}} * 0.74 \frac{{L}_{{CO}_{2}}}{{g}_{Glucose}}$$

These give the following results shown in table 2.

TABLE 2
H₂ and CO₂ production per fresh weight of sample.

| | | FBM1 - | FBM2 - | |
|---|---|---|---|---|
| | COM1 - | Delactose | Soya | FBM3 - Sugar |
| | Wastewater | permeate | molasse | beet molasse |
| ${H}_{2} [ \frac{m {3}_{{H}_{2}}}{m {3}_{FW}} ]$ | 0.43 | 289.7 | 353.8 | 555.1 |
| ${CO}_{2} [ \frac{m {3}_{{CO}_{2}}}{m {3}_{FW}} ]$ | 0.21 | 143.2 | 174.9 | 274.5 |

These results show a great improvement in the production of hydrogen when using a MEC on these high energy substrates compared to wastewater. The higher VS contents shows the greater production of H₂ and CO₂.

To show the same results from a one chambered MEC, where the production would be CH₄ and CO₂—the results from Table 2 are used but in the calculations of conversion of H₂ and CO₂ to CH₄.

4H₂+CO₂→CH₄+2H₂O

A condition for the following simulation is that this conversion has an efficiency of 95%, giving a minor loss in H₂ and thereby CH₄ yield.

This would give the following yields of the substrates:

TABLE 3
CH₄ and CO₂ from a one chambered MEC.

| | | FBM1 - | FMB2 - | |
|---|---|---|---|---|
| | COM1 - | Delactose | Soya | FBM3 - Sugar |
| | Wastewater | permeate | molasse | beet molasse |
| ${CH}_{4} [ \frac{m {3}_{{CH}_{4}}}{m {3}_{FW}} ]$ | 0.10 | 68.8 | 84.0 | 131.8 |
| ${CO}_{2} [ \frac{m {3}_{{CO}_{2}}}{m {3}_{FW}} ]$ | 0.11 | 74.4 | 90.9 | 142.6 |

These yields show the same tendency as seen in Table 2 but to give a more representable view, this could be shown as a ratio, of how much more are produced from the other substrates than for wastewater. With the ratio based on wastewater, this will be 1.

TABLE 4
Ratio between high energy substrates and the production from wastewater.

| | | | | FBM3 - |
|---|---|---|---|---|
| | COM1 - | FBM1 - | FBM2 - | Sugar |
| | Waste- | Delactose | Soya | beet |
| | water | permeate | molasse | molasse |
| $Ratio [ \frac{m {3}_{{CH}_{4}}}{substrate} : \frac{m {3}_{{CH}_{4}}}{wastewater} ]$ | 1 | 667 | 814 | 1278 |

These results show the big effect and difference of using MEC on high energy substrates compared to wastewater, based on fresh weight.

### Example 3—Processing of Biomasses by Anaerobic Digestion and Microbial Electrolysis Cell Processing

The biomasses FBM1-FBM3 from example 1 were used as the first biomass as input to a microbial electrolysis cell processing.

A mixture of biomasses comprising manure, deep litter, and food waste was used as the second biomass.

The system of the invention for processing biomasses first biomass and second biomass separately by microbial electrolysis cell processing and anaerobic digestion, respectively, was found highly suitable with respect to yield of hydrogen gas and biogas. In addition, the easy biodegradability of the first biomass contributed towards a much shorter hydraulic retention time compared to the degradation of the second biomass. This translated to the possibility of producing more biogas by means of an increased processing of the first biomass in the same timeframe.

### Example 4—Hydrogen and Methane Gas Production from a High Energy Substrate in a Dual Chamber Microbial Electrolysis Cell

### Reactor Configuration:

The microbial electrolysis cell reactor (MECR) was a dual chamber microbial electrolysis reactor, with a total capacity of 550 mL in each chamber and separated by a Nafion N117 cation exchange membrane. Both the anode and cathode were made of graphite rod, each with a surface area of 62.5 cm².

### Experiment:

The biomass FBM3—sugar beet molasse was used as the first biomass input in this experiment. The effluent liquid digestate fraction from an anaerobic digester was used as the inoculum source. Additional characteristics of FBM3 are described in the following table.

TABLE 5
Characteristics of the high energy biomass FBM 3-
sugar beet molasse

| | | COD | Nitrogen | Phosphorous | Potassium |
|---|---|---|---|---|---|
| | | (mg/L) | (g/kg) | (g/kg) | (g/kg) |
| FBM3 sugar | beet | 1,323,000 | 6.91 | 0.77 | 30.47 |
| beet molasse | | | | | |

The anodic chamber was first inoculated with 5 mL of FBM3 and 495 mL of the liquid digestate fraction in a volumetric ratio of (1:99). After an initial period of 6 days, 5 mL of the culture was removed and replaced with 5 mL of fresh FMB3 and repeated every 3 days for 4 times in a fed-batch manner until the volumetric ratio of FBM3 has reached approximately (1:19) in the reactor. This stepwise approach ensured that the reactor was not exposed to acidification during the degradation process of FBM3. At the end of the additions, the reactor conditions had reached approximately 122,400 mg/L COD with nitrogen, phosphorous and potassium levels at 4.18 g/kg, 0.85 g/kg and 7.52 g/kg, respectively.

In the cathodic chamber, 150 mL of 0.1 M sodium chloride was added. A cell potential of 0.8 V was applied to the reactor via a power supply and the current was recorded by measuring the voltage drop across an external resistance of 1.3 Ohm. The reactors were gently stirred at 200 rpm and incubated at room temperature for 20 days with separate gas samples taken from the sealed headspace from both the anode and the cathode chambers. The control reactors were treated to the same conditions but without electrodes and without the addition of a cell voltage of 0.8 V. All treatments were performed in duplicates.

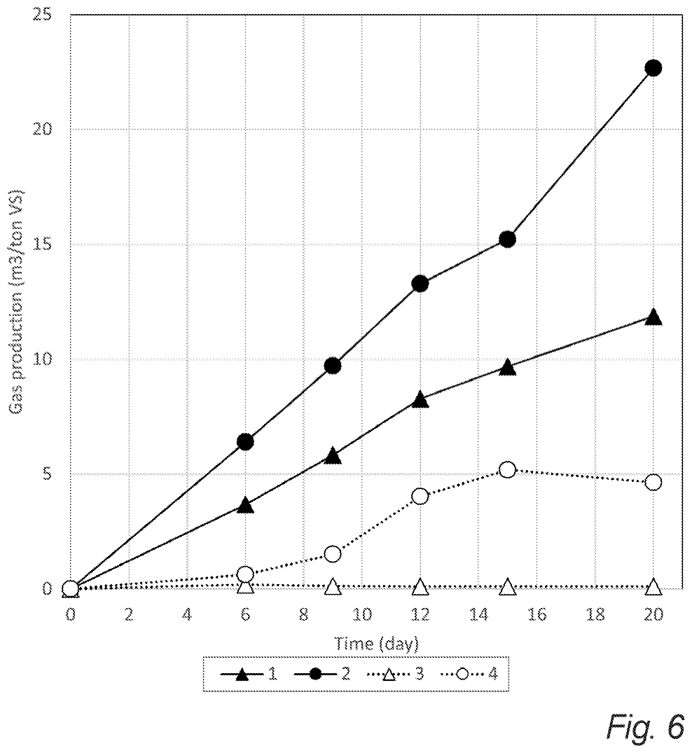

FIG. 6 shows methane and hydrogen production from separate gas streams in dual chamber microbial electrolysis reactors containing FBM3 beet molasses as the first biomass, with and without the addition of 0.8V. Data was from duplicate reactors. Legend: (1) Filled triangle, methane gas from control reactors, (2) filled circle, methane gas from reactors with addition of 0.8 V, (3) open triangle, hydrogen gas from control reactors, (4) open circle, hydrogen gas from reactors with addition of 0.8 V.

FIG. 6 showed that the addition of 0.8V in the MECR produced up to 5.16 m³ H₂/ton VS from the cathodic chamber whereas hydrogen gas was negligible in the control without a voltage supply. Additionally, the MECR yielded 1.9 folds higher methane compared to the controls, suggesting enhanced organic matter degradation due to the added potential. Overall, this corresponded to a 102% increase in energy gained in terms of hydrogen and methane gas in the MECR compared to the control. These results demonstrated the ability of the dual chamber MECR to not only produce hydrogen gas from the first high-energy biomass but also enhance methane production.

### Example 5—Hydrogen and Methane Gas Production from a Mixture of High Energy Substrates in a Dual Chambered Microbial Electrolysis Cell

The MECR configuration used in this experiment was similar to that described above in example 4.

### Experiment:

In this experiment, the possibility of a mixture of two high energy substrates in the MECR was explored to investigate the effect of co-digestion. The two high energy biomasses chosen as the first biomass mixture were FBM1—delactosed permeate and FBM3—sugar beet molasse. The anaerobic digestion effluent liquid digestate fraction was used as the inoculum. Additional characteristics for FBM1 are given in the table below.

TABLE 6
Additional characteristics of FBM1-delactosed permeate

| | COD | Nitrogen | Phosphorous | Potassium |
|---|---|---|---|---|
| | (mg/L) | (g/kg) | (g/kg) | (g/kg) |
| FBM1-delactosed | 343,400 | 2.97 | 5.2 | 29.51 |
| permeate | | | | |

To the anodic chamber of a dual-chamber MECR, 7.5 mL of FBM1, 4 mL of FBM3 and 488.5 ml of liquid digestate fraction were added in the volumetric ratios of (0.8:1. 5:97.7) respectively. After 6 days of incubation, 11.5 mL of reactor liquid was removed and a fresh mixture of the first biomass in the same ratios was added. This was repeated for 3 more times every three days. At the end of the additions, the MECR had reached a COD of approximately 113507 mg/L with nitrogen, phosphorous and potassium levels at 3.5 g/kg, 1.18 g/kg and 9.02 g/kg, respectively. The cathodic chamber configurations follow that described in example 4.

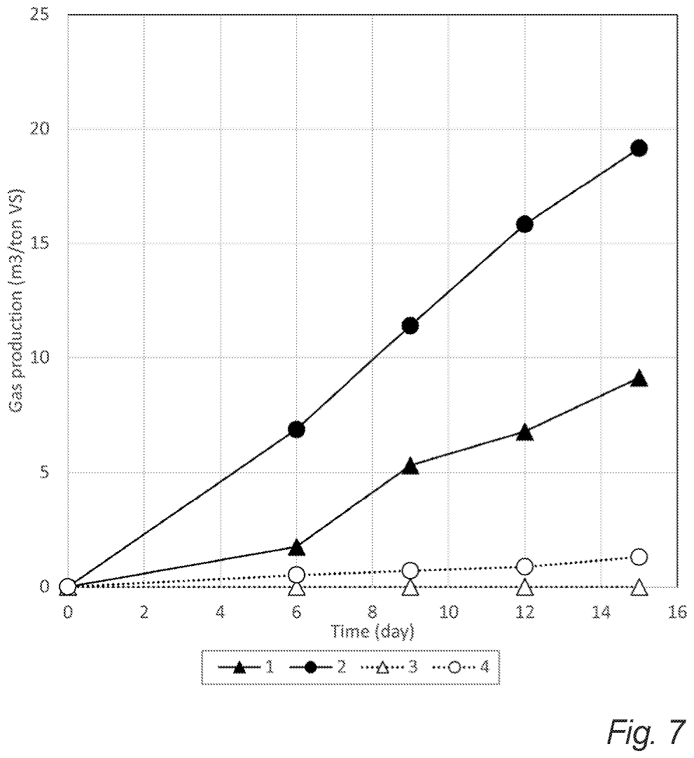

FIG. 7 shows methane and hydrogen production from separate gas streams in dual chamber microbial electrolysis reactors containing a mixture of FBM1 delactosed permeate and FBM3 beet molasses as the first biomass, with and without the addition of 0.8V. Data was from duplicate reactors. Legend: (1) Filled triangle, methane gas from control reactors, (2) filled circle, methane gas from reactors with addition of 0.8 V, (3) open triangle, hydrogen gas from control reactors, (4) open circle, hydrogen gas from reactors with addition of 0.8 V.

According to FIG. 7, hydrogen gas (1.3 m³/ton VS) was produced in the cathodic chamber of the MECR whereas none could be measured for the control reactors. In addition, the MECR produced an additional methane yield of 10.02 m3/ton VS in the anodic chamber compared to the control. Considering the gas production from both chambers, this corresponded to a 110% increase in energy output in the MECR compared to the controls. The results indicate the possibility of the use of high energy substrates such as delactosed permeate and molasses as biomasses in MECR to produced not only hydrogen gas but also enhance methane production.

### Example 6—Methane Gas Production from High Energy Substrate in a Single Chamber Microbial Electrolysis Cell

### Reactor Configuration:

The microbial electrolysis reactor consisted of a single chamber with a total capacity of 550 mL. Both anode and cathode were made of carbon felt with a surface area of 38 cm² and were placed in the same single chamber.

### Experiment:

The biomass FBM3-sugar beet molasse was used in this experiment in the same fed-batch regimen as described earlier in example 4. Briefly, FBM3 and liquid digestate fraction were added step wise until the volumetric ratio has reached (1:19) in a total liquid volume of 200 mL in the reactor.

A cell potential of 0.8 V was applied to the reactors via a power supply and the current was recorded by measuring the voltage drop across an external resistance of 1.3 Ohm. The reactors were incubated at 30° C. for 20 days with gas samples taken from the sealed headspace. The control reactors were treated to the same conditions but without electrodes and without the addition of a cell voltage of 0.8 V. The control treatments were performed in duplicates whereas the MECR were run in quadruplicates.

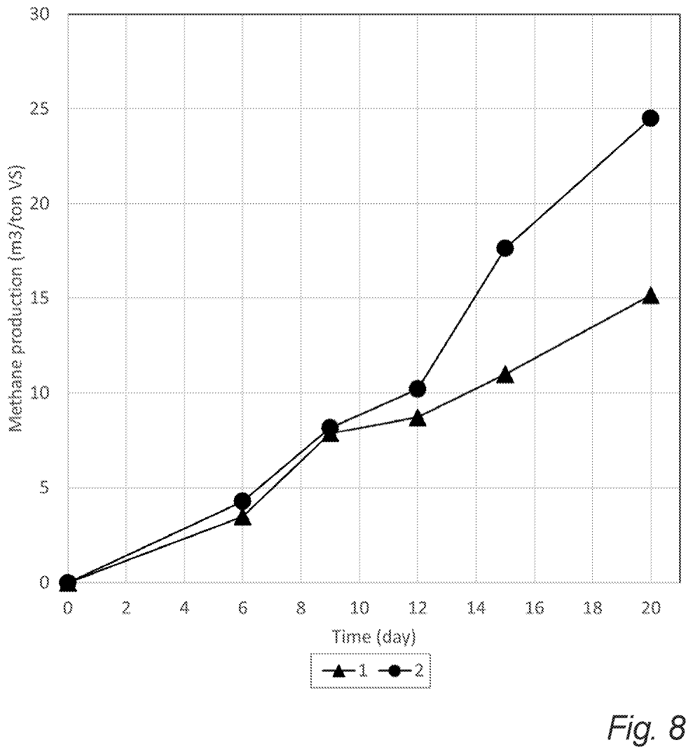

FIG. 8 shows methane production in single chamber microbial electrolysis reactors containing liquid digestate fraction and FBM3 beet molasses, data from duplicate control reactors and quadruplicate microbial electrolysis cell reactors with 0.8V. Legend: (1) Filled triangle, methane gas from control reactors, (2) filled circle, methane gas from reactors with addition of 0.8 V.

According to FIG. 8, the single chamber MECRs poised at 0.8V with FBM3 as substrate produced approximately 61% more methane than control reactors. Hydrogen gas production was not expected as any hydrogen produced at the cathode was likely to have been converted to methane gas by the inoculum present. These results highlight the possibility of using high energy biomass in single-chamber MECR to enhance methane production.

### Example 7—Enhanced Methane Production from Different High Energy Substrates in a Single Chamber Microbial Electrolysis Cell

The single chamber MECR set up was similar to that described earlier in example 6. In this experiment, the potential of other high energy substrates, such as FBM4—fish waste (salmon silage) and FBM5—food waste, for use as first biomass in two separate MECR experiments were explored. The characteristics of the two biomasses are described in the table below.

| | COD | Nitrogen | Phosphorous | Potassium |
|---|---|---|---|---|
| | (mg/L) | (g/kg) | (g/kg) | (g/kg) |
| FBM4-fish waste | 243075 | 9.52 | 2.07 | 2.29 |
| (salmon silage | | | | |
| FBM5-food waste | 419250 | 7.63 | 1.51 | 2.47 |

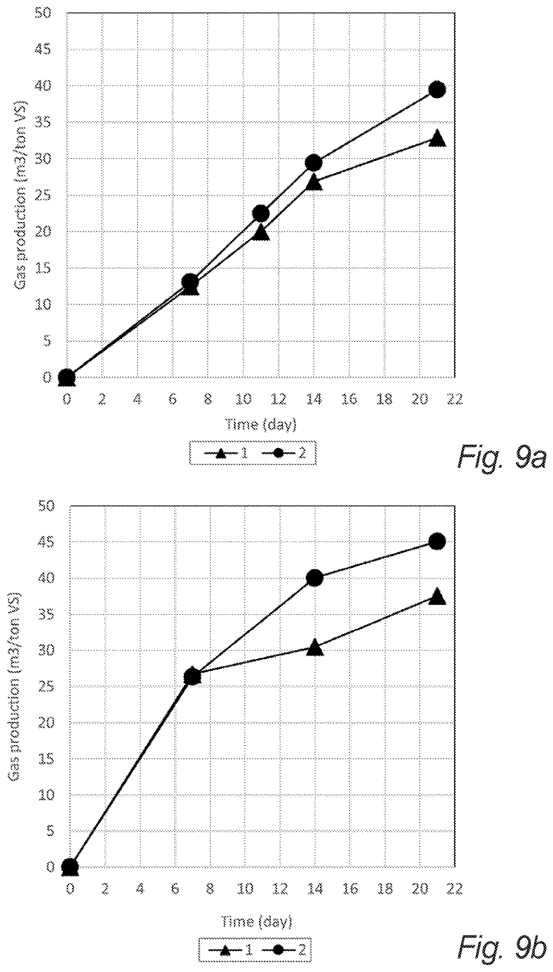

FIG. 9 shows methane production in single chamber microbial electrolysis reactors at 0.8V containing liquid digestate fraction and (a) FBM34 beet molasses fish waste (salmon silage) or (b) FBM 5 food waste, compared to control reactors with no voltage addition. Data from duplicate control reactors and quadruplicate microbial electrolysis cell reactors with 0.8V. Legend: (1) Filled triangle, methane gas from control reactors, (2) filled circle, methane gas from reactors with addition of 0.8 V.

### Experiment 7a—FBM4 Fish Waste

To a set of reactors, approximately 8% v/v or 16 mL of FBM4-fish waste was added at each sampling point (every 7 days) with 184 mL of liquid digestate fraction as the inoculum. An equivalent volume was then removed at each sampling point to keep consistent reactor volumes. This was repeated four times over a period of 14 days until an approximate COD of 102,000 mg/L has been reached with total nitrogen concentrations at 5.92 g/kg, total phosphorus at 0.58 g/kg and total potassium at 4.29 g/kg in the reactors. At the end of the experimental period of 21 days, the methane yield was increased by 20% in the MECR with an addition of 0.8 V compared to the respective controls as seen in FIG. 9a.

### Experiment 7b—FBM5 Food Waste

To a second set of reactors, FBM5-food waste as the first biomass was added at 6% v/v or 12 mL with 188 mL of the liquid digestate fraction as the inoculum. Similarly, 12 mL of reactor liquid was removed at each sampling point (every 7 days) to be replenished with 12 mL of the first biomass. After a period of 14 days, the COD in the reactor has reached approximately 107,000 mg/L with the N, P and K concentrations at 4.15 g/kg, 0.7 g/kg and 5.18 g/kg respectively. FIG. 9b shows that after a lag period of 7 days, the methane yield in the MECR increased steadily until it has reached 20% higher compared to the controls at the end of the experimental period.

These two experiments show the suitability of a variety of different high energy substrates for use as the first biomass in the MECR to enhance methane production

1. A system for producing product gas comprising methane, the system comprising
a first inlet for receiving a first biomass, the first biomass having a dry matter content of at least 15% by weight of the first biomass and a chemical oxygen demand of at least 100,000 mg O2/L, the first biomass having at least one of
a content of nitrogen of at least 4 gram per kg,
a content of phosphorous of at least 5 gram per kg, and
a content of potassium of at least 5 gram per kg,
a second inlet for receiving a second biomass,
a microbial electrolysis cell reactor for processing the first biomass to produce methane and/or hydrogen gas and a first biomass digestate,
an anaerobic digestion reactor for processing the second biomass to produce methane and a second biomass digestate,
a first output for discharging the first biomass digestate,
a second output for discharging the second biomass digestate.

2. The system according to claim 1, wherein the microbial electrolysis cell reactor is configured for producing hydrogen.

3. The system according to claim 1, wherein the microbial electrolysis cell reactor is configured for producing biogas.

4.-5. (canceled)

6. The system according to claim 1, wherein the system is further configured for initializing the microbial electrolysis cell reactor by injecting an initializing biomass comprising filtered second biomass digestate.

7. The system according to claim 1, wherein the system further comprises a biogas upgrader for reducing the content of carbon dioxide in the biogas.

8. The system according to claim 7, wherein the biogas upgrader is connected to microbial electrolysis cell reactor for receiving biogas and/or hydrogen gas and to the anaerobic digestion reactor for receiving biogas.

9. The system according to claim 7, wherein the biogas upgrader is further configured for reducing the content of at least one of hydrogen sulfide, water, and carbon monoxide.

10. The system according to claim 1, wherein the system further comprises a methanation unit configured to convert carbon dioxide from the anaerobic digestion reactor to methane by methanation using hydrogen received from the microbial electrolysis cell reactor.

11. The system according to claim 1, wherein the system is configured to dilute the first biomass before the microbial electrolysis cell reactor.

12-16. (canceled)

17. The system according to claim 1, wherein the system further comprises an error state monitoring system.

18-19. (canceled)

20. The system according to claim 1, wherein the system further comprises a separator for separating a solid fraction from the first biomass.

21. (canceled)

22. The system according to claim 1, wherein the liquid input comprises a backmix feeding line configured to mix a part of the first biomass digestate with the first biomass.

23. (canceled)

24. The system according to claim 22, wherein the liquid input comprises nutrients.

25. The system according to claim 22, wherein the liquid input comprises a liquid second digestate fraction.

26. The system according to claim 1, wherein the microbial electrolysis cell reactor has a capacity of at least 5 m3.

27. The system according to claim 1, wherein the anaerobic digestion reactor has a capacity of at least 500 m3.

28-31. (canceled)

32. The system according to claim 1, wherein the system further comprises a first output storage container configured to receive said first biomass, the first output storage being connected to the first output.

33. The system according to claim 1, wherein the system further comprises a second output storage container configured to receive said second biomass digestate, the second output storage being connected to the second output.

34. The system according to claim 1, wherein the first biomass comprises one or more selected from dairy waste, molasse, fish waste, and food waste.

35. The system according to claim 34, wherein the first biomass further comprises one or more selected from food oils and fats, and glycerin.

36-41. (canceled)

42. The system according to claim 1, wherein the first biomass is free of wastewater.

43. The system according to claim 1, wherein the second biomass has a content of nitrogen, phosphorous, and potassium, each being less than 5 gram per kg.

44. (canceled)

45. The system according to claim 1, wherein the second biomass comprises straw.

46. (canceled)

47. The system according to claim 1, wherein the system further comprises a second microbial electrolysis cell reactor configured to receive the second biomass after processing in said anaerobic digestion reactor, and to feed the second biomass to the second output after processing.

48-56. (canceled)

57. The system according to claim 1, wherein the first outlet and the second outlet are controllable to discharge a predefined amount of the first biomass digestate and the second biomass digestate.

58-60. (canceled)
